(19) United States
(12) Patent Application Publication
(10) Pub. No.: US 2025/0087357 A1
(43) Pub. Date: Mar. 13, 2025

(54) METHOD, APPARATUS, DEVICE AND STORAGE MEDIUM FOR INFORMATION INTERACTION

(71) Applicant: Beijing Zitiao Network Technology Co., Ltd., Beijing (CN)

(72) Inventors: Wei TU, Beijing (CN); Yihang Tang, Beijing (CN)

(21) Appl. No.: 18/882,633

(22) Filed: Sep. 11, 2024

(30) Foreign Application Priority Data
Sep. 11, 2023 (CN) 202311170284.4

(51) Int. Cl. G16H 50/20; G16H 10/20; G16H 40/67

(52) U.S. Cl. CPC G16H 50/20; G16H 10/20; G16H 40/67

(57) ABSTRACT

The embodiment of the disclosure provides method, apparatus, device and storage medium for information interaction. The method includes: in response to a consultation initiating operation, providing an interaction interface of a user and a digital assistant for a health consultation of the user; determining, based on at least one round of conversation between the user and the digital assistant in the interaction interface, whether an information collection requirement for the health consultation is satisfied; and in response to determining that the information collection requirement for the health consultation is satisfied, providing an anticipated health recommendation for the health consultation of the user at the interaction interface. In this way, the digital assistant can more intelligently and effectively provide reasonable health recommendations, which helps to improve user experience.

### CROSS REFERENCE

This application claims priority of the Chinese patent application No. 202311170284.4, entitled “METHOD, APPARATUS, DEVICE AND STORAGE MEDIUM FOR INFORMATION INTERACTION” filed on Sep. 11, 2023, the entire content of which is incorporated herein by reference.

### FIELD

Example embodiments of the present disclosure generally relate to the field of computers, and in particular, to a method, apparatus, device, and computer-readable storage medium for information interaction.

### BACKGROUND

With the development of Internet technologies, it is also desirable in health consultation aspect to enable people to enjoy remote and online services. The online medical improves the supply of high-quality medical resources at high efficiency and low cost, helps the medical institution to expand the radiation range of the medical service, and enables the consultation object to obtain the medical guidance of the authoritative professional at any time. Currently, some websites, terminal applications, etc., attempt to provide online health consultation services. However, in online health consultation, interactivity and expertise are expected to be improved.

### SUMMARY

In a first aspect of the present disclosure, an information interaction method is provided. The method comprises: in response to a consultation initiating operation, providing an interaction interface of a user and a digital assistant for a health consultation of the user; determining, based on at least one round of conversation between the user and the digital assistant in the interaction interface, whether an information collection requirement for the health consultation is satisfied; and in response to determining that the information collection requirement for the health consultation is satisfied, providing an anticipated health recommendation for the health consultation of the user at the interaction interface.

In a second aspect of the present disclosure, an apparatus for information interaction is provided. The apparatus comprises: an interface providing module, configured to in response to a consultation initiating operation, provide an interaction interface of a user and a digital assistant for a health consultation of the user; a condition determining module, configured to determine, based on at least one round of conversation between the user and the digital assistant in the interaction interface, whether an information collection requirement for the health consultation is satisfied; and a suggestion providing module, configured to provide, in response to determining that the information collection requirement for the health consultation is satisfied, provide an anticipated health recommendation for the health consultation of the user at the interaction interface.

In a third aspect of the present disclosure, an electronic device is provided. The device comprises at least one processing unit; and at least one memory coupled to the at least one processing unit and storing instructions for execution by the at least one processing unit. The instructions, when executed by the at least one processing unit, cause the device to perform the method of the first aspect.

In a fourth aspect of the present disclosure, a computer-readable storage medium is provided. The medium stores a computer program, and when the computer program is executed by the processor, the method in the first aspect is implemented.

It should be understood that the content described in this section is not intended to limit the key features or important features of the embodiments of the present disclosure, nor is it intended to limit the scope of the present disclosure. Other features of the present disclosure will become readily understood from the following description.

### BRIEF DESCRIPTION OF DRAWINGS

The above and other features, advantages, and aspects of various embodiments of the present disclosure will become more apparent with reference to the following detailed description taken in conjunction with the accompanying drawings. In the drawings, the same or similar reference numbers refer to the same or similar elements, wherein:

FIG. 1 illustrates a schematic diagram of an example environment in which embodiments of the present disclosure can be implemented;

FIG. 2A to FIG. 2G respectively show schematic diagrams of examples of interaction interfaces according to some embodiments of the present disclosure;

FIG. 3 illustrates a schematic diagram of an example process of prompt verification according to some embodiments of the present disclosure;

FIG. 4A to FIG. 4C respectively show schematic diagrams of examples of interaction interfaces according to some embodiments of the present disclosure;

FIG. 5 illustrates a schematic diagram of an example architecture of a digital assistant application according to some embodiments of the present disclosure;

FIG. 6 shows a flowchart of a process for information interaction according to some embodiments of the present disclosure;

FIG. 7 illustrates a block diagram of an apparatus for information interaction according to some embodiments of the present disclosure; and

FIG. 8 illustrates a block diagram of an electronic device in which one or more embodiments of the present disclosure may be implemented.

### DETAILED DESCRIPTION

Embodiments of the present disclosure will be described in more detail below with reference to the accompanying drawings. While certain embodiments of the present disclosure are shown in the accompanying drawings, it should be understood that the present disclosure may be implemented in various forms, and should not be construed as limited to the embodiments set forth herein, but rather, these embodiments are provided for a more thorough and complete understanding of the present disclosure. It should be understood that the drawings and embodiments of the present disclosure are for exemplary purposes only and are not intended to limit the scope of the present disclosure.

In the description of the embodiments of the present disclosure, the terms “including” and the like should be understood to include “including but not limited to”. The term “based on” should be understood as “based at least in part on”. The terms “one embodiment” or “the embodiment” should be understood as “at least one embodiment”. The term “some embodiments” should be understood as “at least some embodiments”. Other explicit and implicit definition may also be included below.

Herein, unless explicitly stated, “in response to A” performing one step does not imply that this step is performed immediately after “A”, but may include one or more intermediate steps.

It may be understood that the data involved in the technical solution (including but not limited to the data itself, the obtaining, using, storing or deleting of the data) should follow the requirements of the corresponding laws and regulations and related regulations.

It can be understood that before using the technical solutions disclosed in the embodiments of the present disclosure, relevant user should be informed of the types, use ranges, usage scenarios, and the like of the information related to the present disclosure in an appropriate manner according to relevant laws and regulations, and the authorization of the related user may be obtained, wherein the relevant users may include any type of rights body, such as individuals, businesses, and groups.

For example, when an active request of a user is received, prompt information is sent to the related user to explicitly prompt the related user, and the operation requested to be performed will need to obtain and use the information of the related user, so that the related user can autonomously select whether to provide information to software or hardware such as electronic device, application, server or storage medium and etc., executing the operation of the technical solution of the present disclosure according to the prompt information.

As an optional but non-limiting implementation, in response to receiving an active request of a related user, a manner of sending prompt information to the related user may be, for example, a pop-up window, and prompt information may be presented in a text manner in the pop-up window. In addition, the pop-up window may further carry a selection control for the user to select “agree” or “not agree” to provide information to the electronic device.

It may be understood that the foregoing notification and obtaining a user authorization process are merely illustrative, and do not constitute a limitation on implementations of the present disclosure, and other manners of meeting related laws and regulations may also be applied to implementations of the present disclosure.

As used herein, the term “model” may learn an association relationship between respective inputs and outputs from training data such that a corresponding output may be generated for a given input after training is complete. The generation of the model may be based on machine learning techniques. Deep learning is a machine learning algorithm that processes inputs and provides corresponding outputs by using a multi-layer processing unit. The neural network model is one example of a deep learning based model. As used herein, a “model” may also be referred to as a “machine learning model,” a “learning model,” a “machine learning network,” or a “learning network,” which terms are used interchangeably herein.

As discussed above, the online medical improves the supply of high-quality medical resources at high efficiency and low cost, helps the medical institution to expand the radiation range of the medical service, and enables the consultation object to obtain the medical guidance of the authoritative professional at any time. The user may use simple text or voice input, send health and disease related needs to the “digital doctor” of the online medical (i.e., digital assistant), and may perform online health consultation by obtaining a real-time reply of the “digital doctor”. However, the current model based digital assistants, while having powerful natural language processing capabilities, are difficult to deeply optimize for specific business scenarios, such as medical health consultation, due to lack of expertise and logic processing capabilities for a particular field. In addition, traditional models often have difficulties in expansion and access, such as the new medical knowledge and service access requiring extensive manual adjustment and optimization.

In practical applications, various problems exist for interaction between a user and a digital assistant. On the one hand, there is a problem that the consultation question is inconsistent with the consultation object. For example, the user may not explicitly indicate who the consulted object is when consulting, which may cause the digital assistant to not accurately understand the user's needs. On the other hand, there is a problem of high user input threshold. During consultation, a user tends to be confused to medical professional vocabularies and methods of describing conditions, without knowing how to effectively describe problems to digital assistants. This increases the threshold of the user using the digital assistant service, resulting in a decrease in the service effect and affecting the user experience.

According to an embodiment of the present disclosure, an improved solution for information interaction is provided. In this solution, during the interaction process between the user and the digital assistant for health consultation, determine, based on at least one round of conversation between the user and the digital assistant in the interaction interface, whether an information collection requirement for the health consultation is satisfied. In response to determining that the information collection requirement for the health consultation is satisfied, provide an anticipated health recommendation for the health consultation of the user at the interaction interface. In an embodiment of the present disclosure, the digital assistant may collect the health information of the user in an order according to the logic of the doctor's inquiry, and give a professional health recommendation based on this information. In this way, the digital assistant can more intelligently and effectively provide reasonable health recommendations, which helps to improve user experience.

Some example embodiments of the present disclosure will be described in detail below with reference to the accompanying drawings.

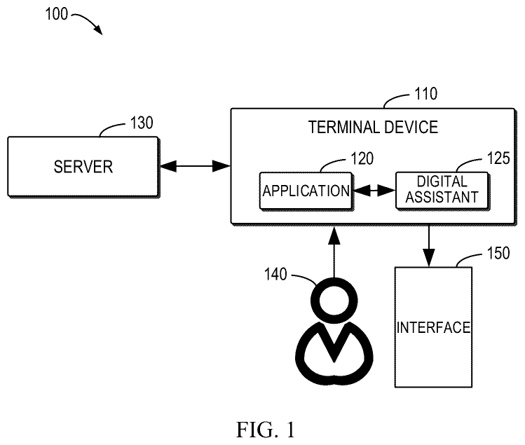

FIG. 1 illustrates a schematic diagram of an example environment 100 in which embodiments of the present disclosure can be implemented. In this example environment 100, an application 120 is installed in the terminal device 110. The user 140 may interaction with the application 120 via the terminal device 110 and/or an attachment device of the terminal device 110.

In some embodiments, the application 120 may be downloaded, installed on the terminal device 110. In some embodiments, the application 120 may also be accessed in other manners, such as web page access etc. In the environment 100 of FIG. 1, if the application 120 is launched, the terminal device 110 may present the interface 150 of the application 120. The interface 150 may include various user interfaces that may be provided by the application 120. Interface 150 is also sometimes referred to as a client interface.

In embodiments of the present disclosure, the application 120 includes any suitable application capable of providing a health consultation service. In some embodiments, the application 120 may implement a dialog interaction with the user 140 based on the digital assistant 125 to provide a health consultation service to the user 140. In some embodiments, the application 120 may provide other services in addition to the interaction of the digital assistant 125, which is not limited in this respect.

In some embodiments, terminal device 110 may communicate with server 130 to enable provision of services to applications 120 and/or digital assistant 125. The terminal device 110 may be any type of mobile terminal, fixed terminal, or portable terminal, including a mobile phone, a desktop computer, a laptop computer, a notebook computer, a netbook computer, a tablet computer, a media computer, a multimedia tablet, a personal communication system (PCS) device, a personal navigation device, a personal digital assistant (PDA), an audio/video player, a digital camera/camcorder, a television receiver, a radio broadcast receiver, an electronic book device, a gaming device, or any combination of the foregoing, including accessories and peripherals of these devices, or any combination thereof. In some embodiments, the terminal device 110 can also support any type of interface for a user (such as a “wearable” circuit, etc.). Applications 130 may be various types of computing systems/servers capable of providing computing power, including, but not limited to, mainframes, edge computing nodes, computing devices in a cloud environment, and so forth.

It should be understood that the structures and functions of the various elements in the environment 100 are described for exemplary purposes only and do not imply any limitation to the scope of the present disclosure.

In an embodiment of the present disclosure, the digital assistant 125 is used to interact with a user. An interaction interface, also referred to as an interaction window or a conversation window, of the user and the digital assistant 125 is presented in the client interface. The digital assistant 125, as an intelligent assistant, has an intelligent conversation and information processing capability. In the interaction interface, the user enters an interaction message, and the digital assistant 125 provides a reply message in response to user input. In application 120, the digital assistant 125 may be launched or awakened in an appropriate manner (e.g., a shortcut, button, or voice) to present an interaction interface with the user. The interaction interface may include an interface element for information interaction, such as an input box, a message list, a message bubble, and the like.

In embodiments of the present disclosure, digital assistant 125 is used to provide services related to health consultation, which may sometimes be referred to as a “digital doctor”. The user may send a health consultation request to the digital assistant 125 by using a simple text or voice input, and may obtain a real-time reply of the digital assistant 125 for medical knowledge science answer, health consultation, or general health management. In this way, the digital assistant 125 can be used to achieve high efficiency and low cost to improve the supply of high-quality medical resources, help the medical institution to expand the radiation range of the medical service, and enable the consultation object to obtain the guidance of the authoritative professional at any time.

In some embodiments, the digital assistant 125 may determine a reply to the user's question by means of a model. In some embodiments, the model may be trained based on a general corpus and a specialized corpus for the medical field, thereby enabling reachable, credible, understandable medical knowledge publicity and health consultation guidance. The specialized corpus for the medical field may include, for example, national and international authoritative clinical guidelines, documents, textbooks, clinical doctor-patient communication question-answer data, and the like. In some embodiments, the training of the medical safety related mechanism can be strengthened during the model training, so that the model can have the capability of providing credible health consultation recommendations, and a comprehensive safety test is passed. In some embodiments, when the performance of the digital assistant 125 is evaluated, the capability of the digital assistant 125 in terms of communication expression capability and humanistic care and friendliness may also be evaluated, so that the digital assistant 125 can implement aspects such as a empathy with a consultation object, understanding the need of the consultation object, detailed and patient communication, and the like, which is conducive to alleviating the problems of short communication time and insufficient communication.

The model may be deployed on the terminal device 110 or may be accessed by the terminal device 110. Alternatively, the terminal device 110 may communicate with the model to provide the application management service for the user 140 with the model. For example, the terminal device 110 may send a prompt input to the model based on the interaction information between the user 140 and the digital assistant 125, and obtain a processing result of the model. As an alternative or as an addition, the digital assistant 125 itself may also be acquired based on a model. The digital assistant 125 may process the session message input by the user 140 to obtain a requirement of the user 140, for example, a problem expected to be consulted by the user 140, and the like. The model may be configured locally or independently of terminal device terminal device 110 according to requirements, and the present disclosure is not intended to be limited thereto.

In an embodiment of the present disclosure, in response to the consultation initiating operation, an interactive interface of the user 140 and the digital assistant 125 is provided for the health consultation of the user 140. The consultation initiation operation may be an operation initiated by the user 140 in any suitable manner. The health consultation may be a health consultation with the user 140 as a consulting object, or may be other advisory object specified by the user 140. In the interactive interface between the user 140 and the digital assistant 125, various types of interactive messages may be supported by the user through text or voice input. The voice input may be converted to a text message. The digital assistant 125 determines a corresponding reply based on the text entered by the user.

In some cases, the user 140 may input a file in a non-text message format for consultation. In an embodiment of the present disclosure, an interaction with a message of a multimodal data format input by the user is supported, including a text message format and a non-text message format. The digital assistant 125 can also properly determine the reply when the user 140 inputs the file in the non-text message format.

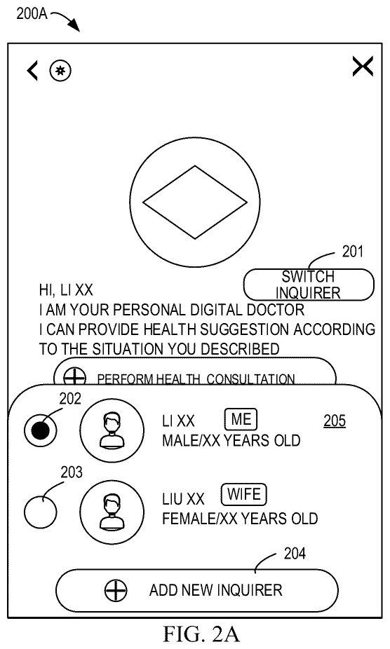

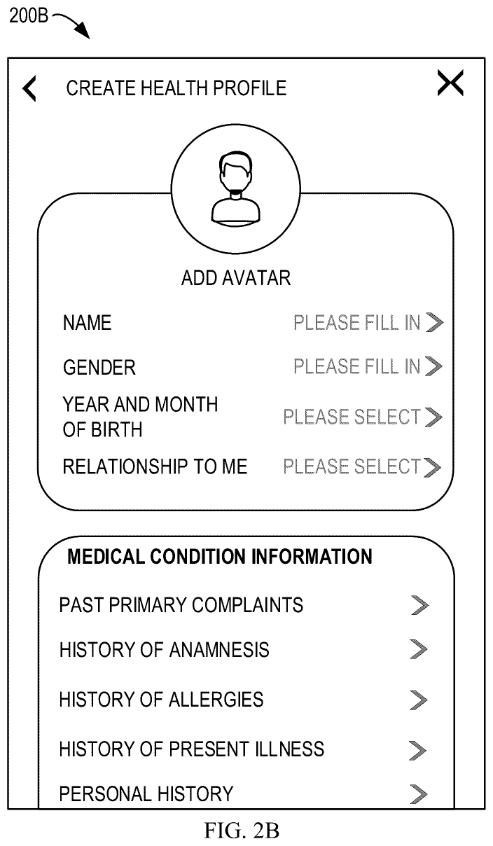

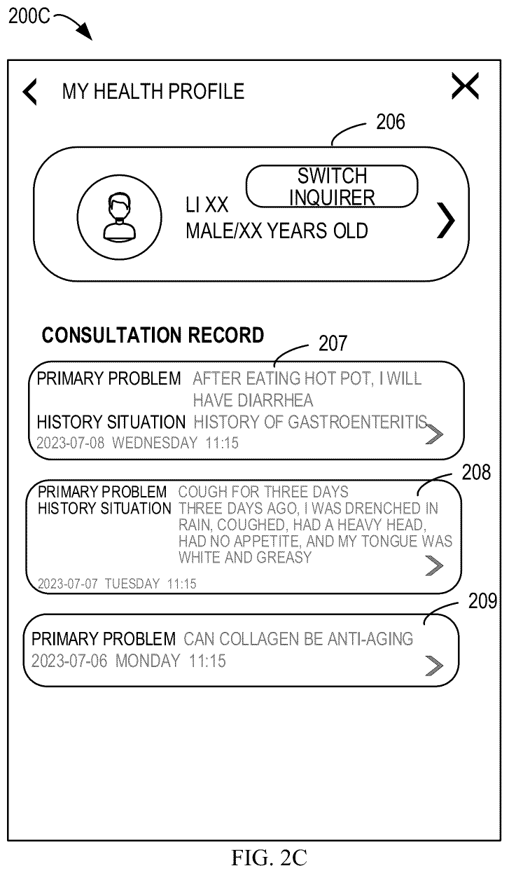

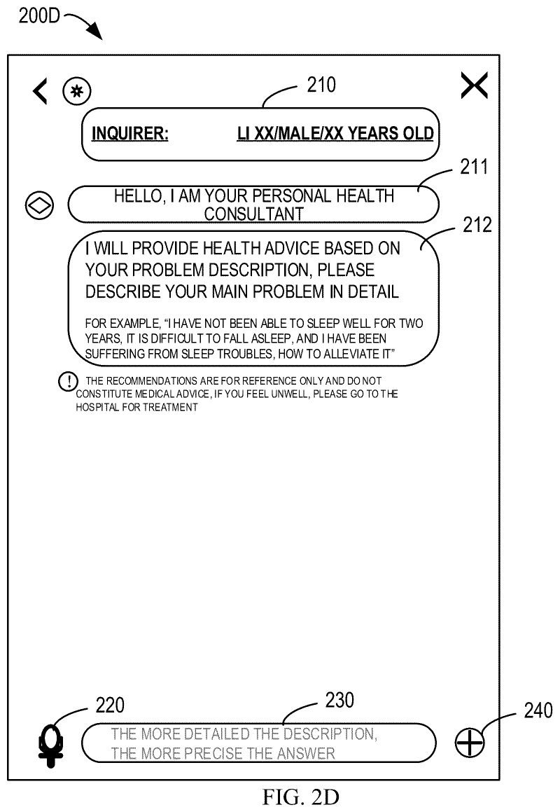

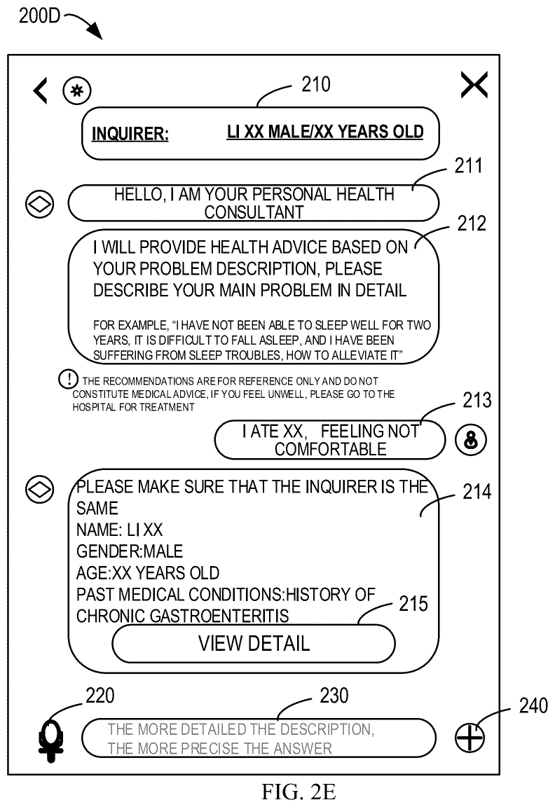

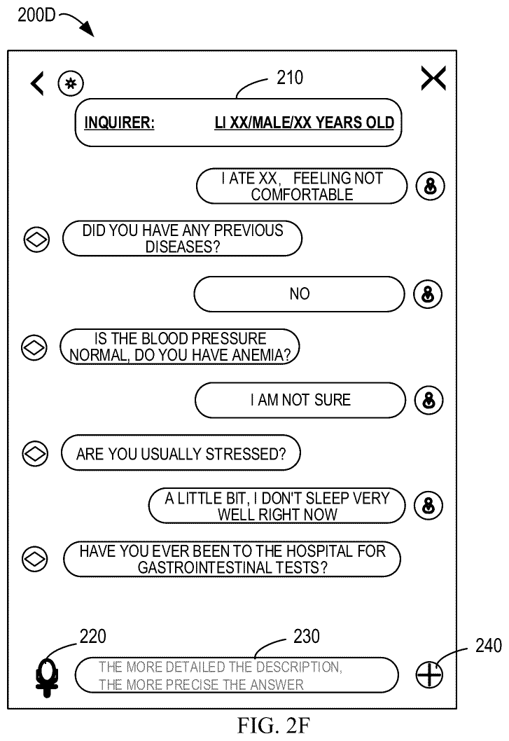

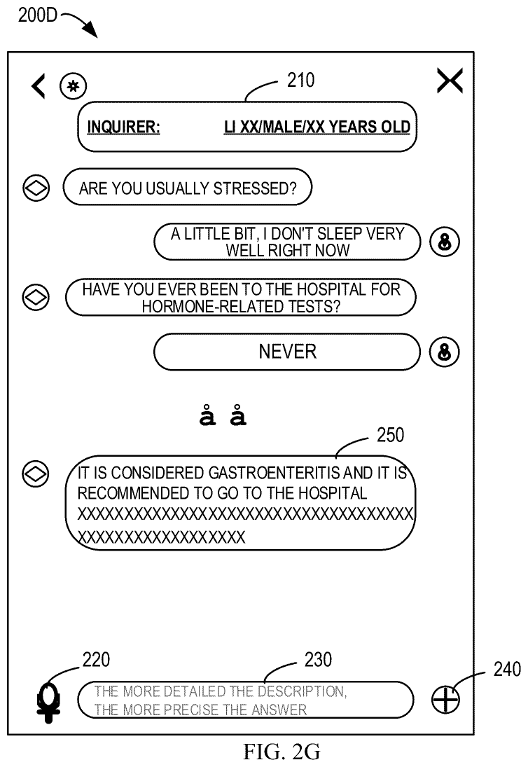

In the following, for a better understanding of the embodiments of the present disclosure, reference will be made to the examples shown in FIG. 2A to FIG. 5 for description. It should be understood that these figures are merely exemplary, for example, various interface designs may actually exist for the interface. Individual graphical elements in the interface may have different arrangements and different visual representations, one or more of the elements may be omitted or replaced, and one or more other elements may also be present. Embodiments of the present disclosure are not limited in this respect.

In some embodiments, the terminal device 110 may provide an interaction interface for selecting an inquirer. Such an interaction interface may be, for example, the interaction interface 200A shown in FIG. 2A. The interaction interface 200A includes an inquirer selection control 201. The terminal device 110, for example, may present the inquirer selection page 205 in response to detecting a selection operation on the inquirer selection control 201. The inquirer selection page 205 includes all inquirers that have been created. As shown in FIG. 2A, the inquirer selection page 205 presents a inquirer Liu XX and a inquirer Liu XX. The inquirer selection page 205 also presents a gender, an identification (e.g., avatar), an age, a relationship (e.g., himself or herself, parent, spouses, child, etc.) between the inquirer and the current user (i.e. user 140) corresponding to each inquirer. For example, the inquirer selection page 205 presents a inquirer Liu XX, a gender of female, XX years old, and is current user's wife (that is, the wife of Li XX, Liu XX, and Li XX are in a spouses relationship). The terminal device 110 may determine, in response to receiving the selection operation on the control (for example, the control 202 and the control 203) corresponding to the different inquirers in the inquirer selection page 205, that the confirmation for the target consultation object is received, and then determine that the inquirer corresponding to the control is the target consultation object (that is, the object of the health consultation) for the health consultation. For example, the terminal device 110 may determine, in response to detecting the selection operation on the control 202, that the inquirer Li XX is the target consultation object for the health consultation.

In some embodiments, the terminal device 110 may further determine, in response to detecting the selection operation on the control 204, that a new inquirer is to be created. The terminal device 110 may present an interaction interface 200B for creating a new inquirer as shown in FIG. 2B. The terminal device 110 may determine the related information of the newly created inquirer based on the user input received in the interaction interface 200B. For example, the terminal device 110 may determine the name of the newly created inquirer based on the user input received at the “name” in the interaction interface 200B. In some embodiments, the newly created inquirer will be presented in the inquirer selection page 205 shown in FIG. 2A.

In some embodiments, the terminal device 110 may also present the interaction interface 200C shown in FIG. 2C in response to a viewing operation on the inquirer (e.g., a selection operation on a certain inquirer (e.g., the inquirer Li XX) in the inquirer selection page 205). The interaction interface 200C presents identification information 206 (including an avatar, name, a gender, etc. of the candidate Li XX) of the selected candidate (i.e., candidate Li XX). Historical consultation records 207, 208, 209 of the candidate Li XX is also presented in the interaction interface 200C. In some embodiments, the terminal device 110 may present, in response to detecting a selection operation on a certain historical consultation record, an interaction interface corresponding to the historical consultation record. For example, the terminal device 110 may present, in response to detecting a selection operation on the historical consultation record 207, a historical interaction interface between the user 140 and the digital assistant 125, and the historical interaction interface presents a multi-round conversation about the historical consultation record 207.

In some embodiments, after determining the inquirer to be subjected to the health consultation, the terminal device 110 may provide the interaction interface 200D shown in FIG. 2D in response to a consultation initiating operation (for example, a selection operation on this control “perform health consultation” in FIG. 2A, or a selection operation on a certain inquirer in the inquirer selection page 205). Interaction interface 200D is an interaction interface between user 140 and digital assistant 125, which may be used for health consultation of the user 140. In some embodiments, the terminal device 110 may display the identification information of the target consultation object in a predetermined area of the interaction interface 200D. The predetermined area herein may be any area (for example, above, left, right, upper left corner, upper right corner, etc.) in the interaction interface 200D, which is not limited in the present disclosure. In some embodiments, in order to enable the user 140 to more intuitively observe the information of the target consultation object, and to reduce the influence of the predetermined area on the session presentation area (i.e., the area presenting the conversation between the digital assistant 125 and the user 140), the predetermined area may be the upper area of the interaction interface 200D. The target consultation object herein is an object of the health consultation, that is, the selected inquirer (for example, the inquirer Li XX).

In some embodiments, in response to the interaction interface 200D being presented, the terminal device 110 may present a session message (e.g., session message 211 and session message 212) from digital assistant 125 in interaction interface 200D. The session message here is a message presented before the user 140 is issuing a query in the interaction interface 200D, such a message may be used to introduce the functionality of the digital assistant 125 and to instruct the user 140 on how to perform the conversation with the digital assistant 125. In some embodiments, the interaction interface 200D includes input controls 220, 230, and 240. The terminal device 110 may, for example, in response to detecting a selection operation on the input control 220, receive the speech of the user 140, and convert the speech into text content to be presented in the interaction interface 200D. For example, the terminal device 110 may present, in response to detecting a selection operation on the input control 230, an input entrance of the message, and then present the text content input by the user 140 received at the input entrance in the interaction interface 200D. For example, the terminal device 110 may also present a file selection page (for example, an image selection page) in response to detecting a selection operation on the input control 240, and further present at least one file selected in the file selection page (for example, at least one image) in the interaction interface 200D.

In some embodiments, in response to the consultation initiating operation (e.g., the terminal device 110 may determine that a consultation initiating operation from the user 140 is received in response to detecting the session message 213 from the user 140), the terminal device 110 may provide a confirmation request for the target consultation object. As shown in FIG. 2E, the terminal device 110 may provide a confirmation request for the target consultation object to the user 140 by providing a session message 214 including the inquirer related information and the control 215. The terminal device 110 may determine that the object of the health consultation is an object in the session message 214, that is, determine that the Li XX is the target consultation object, in response to receiving an confirmation for the target consultation object (for example, receiving such a session message from the user 140 that “confirms the consistency”). In some embodiments, the terminal device 110 may present detailed information of the inquirer (i.e., Lie XX) included in the session message 214 in response to detecting a selection operation on the control 215 (e.g., may present detailed information of the inquirer by presenting the interaction interface 200C shown in FIG. 2C).

In an embodiment of the present disclosure, the user 140 and the digital assistant 125 may perform a multi-round conversation in the interaction interface 200D. As shown in FIG. 2F, the interaction interface 200D includes a multi-round conversation between the user 140 and the digital assistant 125. The terminal device 110 may determine, based on at least one round of conversations between the user 140 and the digital assistant 125 in the interaction interface 200D, whether the information collection requirement for the health consultation is satisfied. The information collection requirement herein may be, for example, whether a symptom (e.g., occurrence/duration, extent, frequency of pain etc.) is included, disease history (e.g., past medical history, surgical history, family history, etc.), personal characteristics/medical history (e.g., gender/age, allergy history/drug history, lifestyle, etc.), physical examination (e.g., body temperature, reflection/lump, organ/feel, etc.), laboratory/imaging study examination (e.g., blood/urine/tissue, biochemical, X-ray/CT/nuclear magnetic resonance, etc.), etc. For example, the terminal device 110 may determine, in response to obtaining the information through at least one round of conversation, that the information collection requirement is satisfied. For example, the terminal device 110 may determine, in response to not obtaining the information or only some of the information is obtained, that the information collection requirement is not satisfied.

In a scenario that it is determined that the information collection requirement for the health consultation is not satisfied, the terminal device 110 may provide a guidance reply of the digital assistant 125 in the interaction interface 200D to guide the user 140 to provide at least one type of health information. The at least one type of health information may be, for example, at least one type of health information mentioned in the foregoing information collection requirement. For example, when the information collection requirement is not satisfied, the terminal device 110 may provide the query information of the information required by the digital assistant 125 for the health consultation. Such query information may guide the user 140 to reply to the question of the digital assistant 125 to enable the terminal device to obtain further information input by the user 140 for the health consultation. In scenario where it is determined that the information collection requirement for the health consultation is satisfied, the terminal device 110 may provide, in the interaction interface 200D, an anticipated health recommendation for the health consultation of the user. As shown in FIG. 2G, in response to determining that the information collection requirement for the health consultation is satisfied, the terminal device 110 may provide the anticipated health recommendation 250 for the health consultation of the user 140 at the interaction interface 200D.

Regarding the reply message from the digital assistant 125 for the user 140 in the interaction interface 200D, in some embodiments, such a reply message is a reply message generated by the terminal device 110 based on the query message from the user 140. Specifically, in response to receiving the first query of the user 140, the terminal device 110 may generate, based on at least the first query (for example, based on at least one round of conversation prior to the first query and the historical consultation record of the target consultation object), a first reply for the first query, and provide the first reply in the interaction interface 200D.

In some embodiments, the terminal device 110 may utilize the trained language model to generate a first reply for the first query. Specifically, the terminal device 110 may generate the prompt input corresponding to the first query based on at least the prompt bank for the digital assistant 125. The prompt bank herein may include a plurality of prompt templates conforming to the information collection requirement described above. The terminal device 110 may generate the prompt input corresponding to the first query based on the first query and the prompt template in the prompt bank. For example, if the first query is “I ate XX, feeling not comfortable”, the terminal device 110 may determine that the first query is a query for the symptom, and then obtain the prompt template associated with the symptom from the prompt bank (for example, “XXXXX, why?”). The terminal device 110 may generate a prompt input (for example, generate a prompt input “I eat XX, feeling not comfortable, why?”) based on the prompt template matching the first query obtained from the prompt bank.

The terminal device 110 may provide the prompt input to the model to obtain the output of the model. The model herein may be, for example, a model obtained by using a long chain technology. The long chain technology can improve the expandability and access capability of the model, can realize rapid iteration and expansion of the model, enable new medical knowledge and services to quickly access, and continuously improve the quality and range of model application.

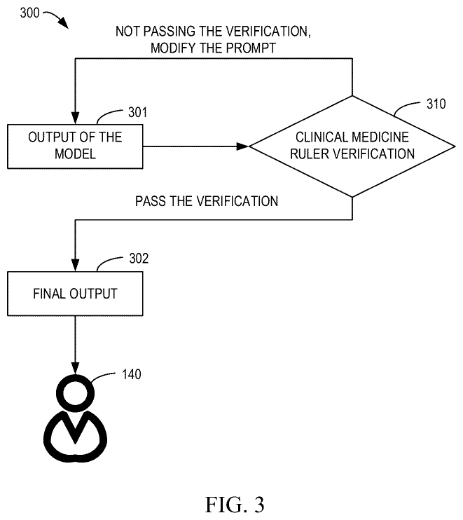

The terminal device 110 may determine the first reply based on the output of the model. The output of the model here may be, for example, a reply to the health consultation given by the digital assistant 125 for the first query of the user 140. In some embodiments, the output of the model is the anticipated health recommendation from the digital assistant 125 in the interaction interface 200D for the health consultation of the user 140. In some embodiments, to ensure the reasonability and accuracy of the generated first reply, the terminal device 110 may further perform verification on the output of the model. FIG. 3 illustrates a schematic diagram of an example process 300 of the prompt verification according to some embodiments of the present disclosure. As shown in FIG. 3, after obtaining the output 301 of the model, the terminal device 110 may perform clinical medicine ruler verification on the output of the model, and determine whether the output of the model may pass the verification.

Specifically, the terminal device 110 may determine whether the output of the model satisfies a predetermined medical verification rule. For example, if the output of the model indicates the user to uses both the A drug and the B drug at the same time but the clinical medicine prescribes that the two drugs cannot be used at the same time, thus the terminal device 110 can determine that the output of the model does not satisfy the predetermined medical verification rule, that is, the output of the model cannot pass the verification of the clinical medicine rule.

In response to the output of the model not satisfying the medical verification rule, the terminal device 110 determines that the quality of the model output corresponding to the prompt input currently used is relatively poor, and the terminal device 110 may modify the prompt input. For example, the terminal device 110 may determine other prompt template from the prompt bank, and generate a modified prompt input based on the other prompt template and the first query. The terminal device 110 may provide the modified prompt input to the model to obtain other output of the model and determine the first reply based on the other output of the model. In this way, the terminal device 110 may perform the process of generating the prompt input—obtaining the input of the corresponding model—performing verification on the output for a plurality of times, until the output of the model pass the verification of the clinical medicine ruler. When the output of the model passes the verification of the clinical medicine ruler, the terminal device 110 may determine that the output of the model satisfies the medical verification rule, and such output of the model is the final output 302 of the model. The terminal device 110 may determine a first reply for the user 140 based on the final output 302 (i.e., the output of the model that satisfies the medical verification rule). In this way, the terminal device 110 may ensure the accuracy of the first reply that is finally provided to the user by performing verification on the output of the model, which helps to improve the user experience of performing health consultation by the user.

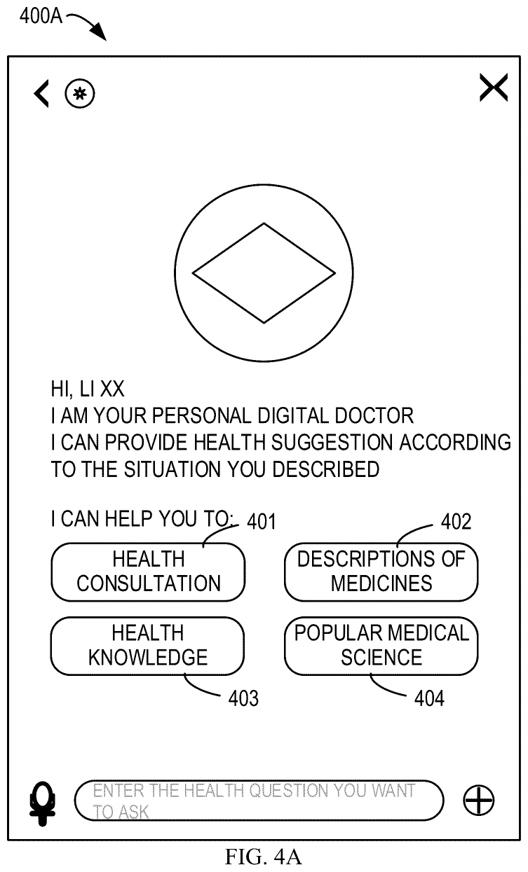

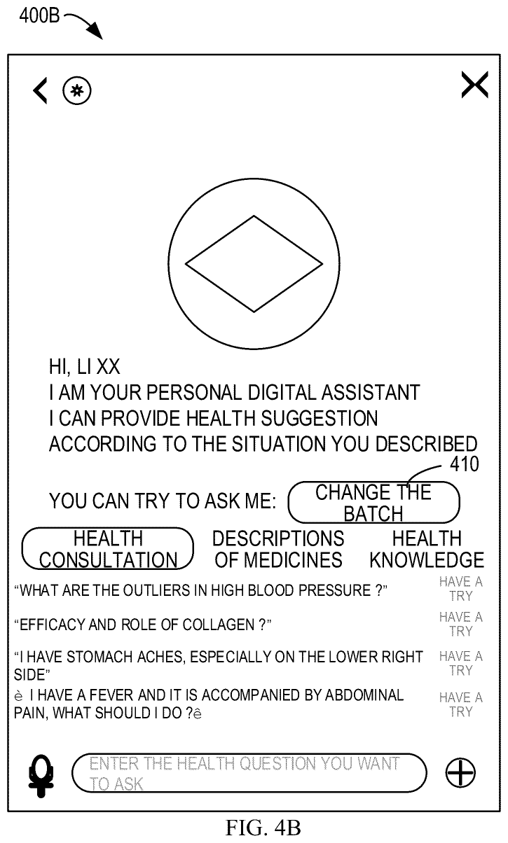

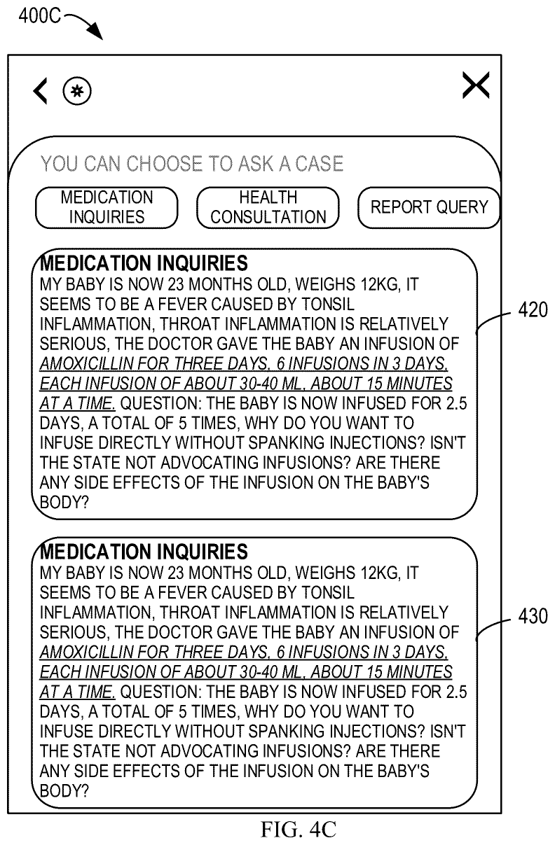

Considering that there is a scenario in which the user 140 may not be adept to question and answer with the digital assistant 125 (e.g., the user 140 is unable to accurately describe the condition and what health recommendation is desired), in some embodiments, the terminal device 110 may also provide a query example to the user 140 to indicate how the user 140 issues a query to the digital assistant 125. Specifically, in some embodiments, the terminal device 110 may provide at least one category label of the query example in an interaction interface between the user 140 and the digital assistant 125. As shown in FIG. 4A, the terminal device 110 may present an interaction interface 400A as shown in FIG. 4A. A plurality of category labels 401, 402, 402, and 404 of the query example are presented in the interaction interface 400A. In some embodiments, the terminal device 110 may determine that the target category label is selected in response to detecting a selection operation on the target category label, and further provide at least one query example under the target category label. Such an query example may be, for example, a simple question. For example, the terminal device 110 may determine, in response to detecting the selection operation on the category label 401, that the category label is the selected target label, and then present the interaction interface 400B shown in FIG. 4B. The terminal device 110 provides at least one query example corresponding to the target category label (that is, the category label 401) in the interaction interface 400B. In some embodiments, the terminal device 110 further presents a control 410 in the interaction interface 400B. The terminal device 110 may replace at least part of the at least one query example under the target category label provided in the interaction interface 400B in response to detecting the selection operation on the control 410.

In some embodiments, the terminal device 110 may further switch to present at least one query example corresponding to another category label in response to detecting a selection operation on another category label other than the target category label presented in the interaction interface 400B. In this case, the another category label selected may be considered a new target category label. For example, in the interaction interface 400B, the terminal device 110 may determine, in response to detecting a selection operation on the category label “description of medicines”, that the switched category label “description of medicines” is a new target category label, and present, at the interaction interface 200B, at least one query example corresponding to the category label “description of medicines”.

In some embodiments, the query example may also be a more complex description text including symptoms, health consultation schemes, and the like. For example, the terminal device 110 may further present an interaction interface 400C as shown in FIG. 4C. The interaction interface 400C includes at least one query example, including example 420 and example 430. Certainly, more examples may also be included, which is not limited in the present disclosure. The query example shown in the interaction interface 400C is no longer simply a simple question but description text containing symptoms, health consultation schemes, and detailed questions. It may be understood that, in the interaction interface 400C, the terminal device 110 may also determine, in response to detecting the selection operation on a new category label, that the new category label is the new target category label, and present at least one query example corresponding to the new target category label on the interaction interface 200C.

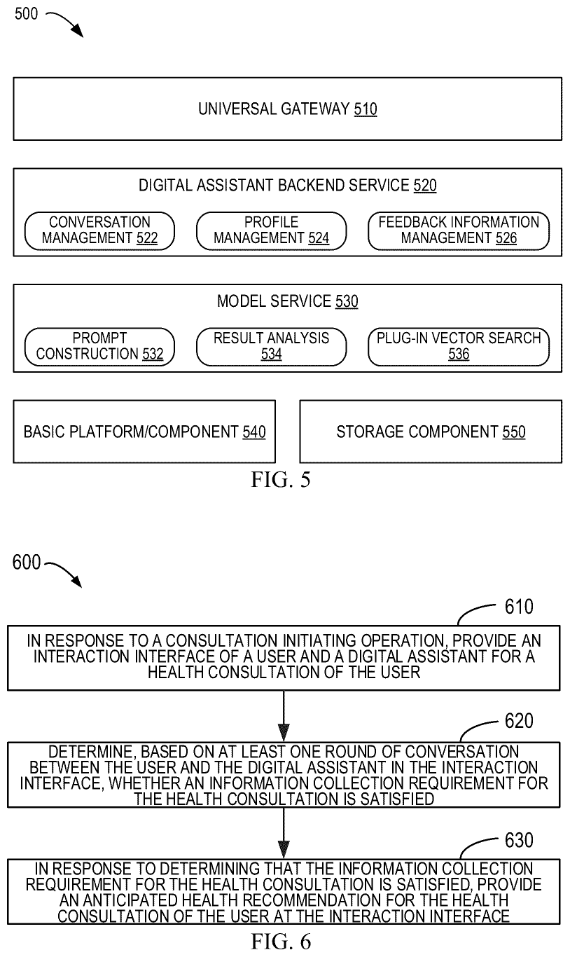

FIG. 5 illustrates a schematic diagram of an example architecture 500 of a digital assistant application according to some embodiments of the present disclosure. The architecture 500 includes a universal gateway 510, a digital assistant backend service 520, a model service 530, a basic platform/component 540, and a storage component 550. The universal gateway 510 is configured to communicate with other electronic devices. The digital assistant backend service 520 is configured to provide functions such as conversation management 522, profile management 524, and feedback information management 526 to the user. The conversation management 522 is configured to manage conversation information between the digital assistant 125 and the user 140 in the interaction interface, the profile management 524 is configured to manage profile information of the at least one inquirer, and the feedback information management 526 is configured to manage a satisfaction degree and/or evaluation information of the reply information provided by the user 140 on the digital assistant 125. Model service 530 includes functions such as prompt construction 530, result analysis 534, plug-in vector search 536, and the like. The prompt construction 530 is used to generate and modify the prompt input. The result analysis 534 may also be referred to as a structured result analysis for presenting health recommendations or the like to the user 140 in a manner of structured data. The basic platform/component 540 and the storage component 550 may be collectively referred to as a basic building layer of the architecture 500 for building the underlying architecture of the application 120 corresponding to the digital assistant 125.

In summary, according to example embodiments of the present disclosure, the digital assistant may collect the health information of the user orderly according to the logic of the doctor's inquiry, and give a professional health recommendation based on this information. In this way, the digital assistant can more intelligently and effectively provide reasonable health recommendations, which helps to improve user experience.

FIG. 6 shows a flowchart of a process 600 for information interaction according to some embodiments of the present disclosure. Process 600 may be implemented at terminal device 110. The process 600 is described below with reference to FIG. 1.

At block 610, the terminal device 110, in response to a consultation initiating operation, provides an interaction interface of a user and a digital assistant for a health consultation of the user.

In block 620, the terminal device 110 determines, based on at least one round of conversation between the user and the digital assistant in the interaction interface, whether an information collection requirement for the health consultation is satisfied.

At block 630, the terminal device 110 in response to determining that the information collection requirement for the health consultation is satisfied, provides an anticipated health recommendation for the health consultation of the user at the interaction interface.

In some embodiments, the process 600 further includes, in response to determining that the information collection requirement for the health consultation is not satisfied, providing a guidance reply of the digital assistant to guide the user to input further information for the health consultation.

In some embodiments, the process 600 further includes: in response to receiving a first query of the user, generating a first reply to the first query based at least on the first query; and providing the first reply at the interaction interface.

In some embodiments, generating the first reply includes: generating a prompt input corresponding to the first query based at least on a prompt bank for the digital assistant; providing the prompt input to a model to obtain an output of the model; and determining the first reply based on the output of the model.

In some embodiments, determining the first reply based on the output of the model includes: determining whether the output of the model satisfies a predetermined medical verification rule; and in response to the output of the model satisfying the medical verification rule, determining the first reply based on the output of the model.

In some embodiments, the process 600 further includes: in response to the output of the model failing to satisfy the medical verification rule, modifying the prompt input; providing the modified prompt input to the model to obtain a further output of the model; and determining the first reply based on the further output of the model.

In some embodiments, the process 600 further includes: providing at least one category label of an query example at the interaction interface; and providing, based on a target category label in the at least one category label being selected, at least one query example under the target category label at the interaction interface.

In some embodiments, a reply of the digital assistant in the at least one round of conversation is used to guide the user to provide at least one class of health information.

In some embodiments, the model is trained based on a general corpus and a specialized corpus for medical field.

In some embodiments, providing the interaction interface includes: displaying identification information of a target consultation object in a predetermined area of the interaction interface, an object of the health consultation being the target consultation object.

In some embodiments, the process 600 further includes: in response to the consultation initiating operation, providing a confirmation request for the target consultation object; and in response to receiving the confirmation for the target consultation object, determining that the object of the health consultation is the target consultation object.

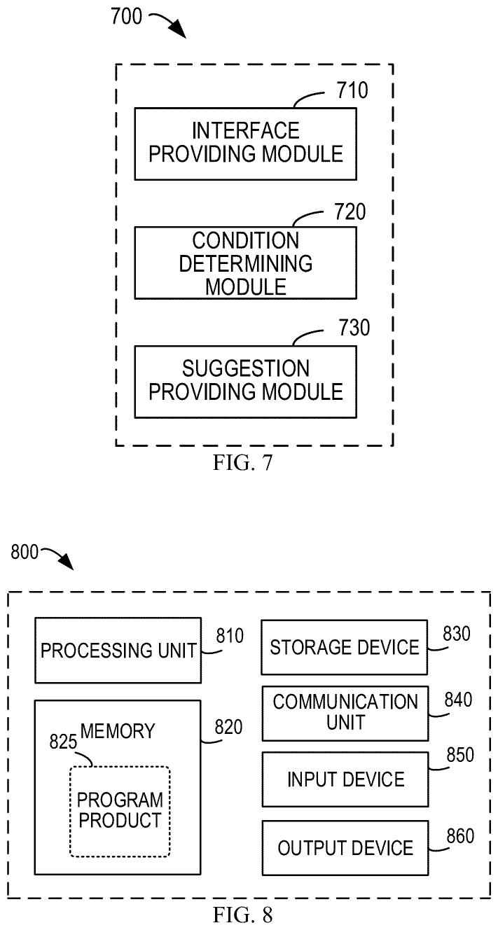

Embodiments of the present disclosure also provide a corresponding apparatus for implementing the above method or process. FIG. 7 is a schematic structural block diagram of an apparatus 700 for information interaction according to some embodiments of the present disclosure. The apparatus 700 may be implemented or included in the terminal device 110. The various modules/components in the apparatus 700 may be implemented by hardware, software, firmware, or any combination thereof.

As shown in FIG. 7, the apparatus 700 includes an interface providing module 710, configured to in response to a consultation initiating operation, provide an interaction interface of a user and a digital assistant for a health consultation of the user. The apparatus 700 further includes a condition determining module 720, configured to determine, based on at least one round of conversation between the user and the digital assistant in the interaction interface, whether an information collection requirement for the health consultation is satisfied. The apparatus 700 further includes a suggestion providing module 730, configured to provide, in response to determining that the information collection requirement for the health consultation is satisfied, provide an anticipated health recommendation for the health consultation of the user at the interaction interface.

In some embodiments, the apparatus 700 further includes: a guidance reply providing module configured to, in response to determining that the information collection requirement for the health consultation is not satisfied, provide a guidance reply of the digital assistant to guide the user to input further information for the health consultation.

In some embodiments, the apparatus 700 further includes: a first reply generation module, configured to in response to receiving a first query of the user, generate a first reply to the first query based at least on the first query; and first reply providing module, configured to provide the first reply at the interaction interface.

In some embodiments, the first reply generation module includes: a prompt generation module configured to generate a prompt input corresponding to the first query based at least on a prompt bank for the digital assistant; a first model input module configured to provide the prompt input to a model to obtain an output of the model; and a first reply determining module configured to determine the first reply based on the output of the model.

In some embodiments, the first reply determining module includes: an output judging module configured to determine whether the output of the model satisfies a predetermined medical verification rule; and a determining module configured to in response to the output of the model satisfying the medical verification rule, determine the first reply based on the output of the model.

In some embodiments, the apparatus 700 further includes: a prompt modification module configured to in response to the output of the model failing to satisfy the medical verification rule, modify the prompt input; a second model input module configured to provide the modified prompt input to the model to obtain a further output of the model; and a second reply determining module configured to determine the first reply based on the further output of the model.

In some embodiments, the apparatus 700 further includes: a label providing module configured to provide at least one category label of an query example at the interaction interface; and an example providing module configured to provide, based on a target category label in the at least one category label being selected, at least one query example under the target category label at the interaction interface.

In some embodiments, a reply of the digital assistant in the at least one round of conversation is used to guide the user to provide at least one class of health information.

In some embodiments, the model is trained based on a general corpus and a specialized corpus for medical field.

In some embodiments, the interface providing module 710 includes: an information display module configured to display identification information of a target consultation object in a predetermined area of the interaction interface, an object of the health consultation being the target consultation object.

In some embodiments, the apparatus 700 further includes: a request providing module configured to in response to the consultation initiating operation, provide a confirmation request for the target consultation object; and a consultation object confirmation module configured to, in response to receiving the confirmation for the target consultation object, determine that the object of the health consultation is the target consultation object.

The units and/or modules included in the apparatus 700 may be implemented in various manners, including software, hardware, firmware, or any combination thereof. In some embodiments, one or more units and/or modules may be implemented using software and/or firmware, such as machine-executable instructions stored on a storage medium. In addition to or as an alternative to machine-executable instructions, some or all of the units and/or modules in the apparatus 700 may be implemented, at least in part, by one or more hardware logic components. By way of example and not limitation, exemplary types of hardware logic components that may be used include field programmable gate arrays (FPGAs), application specific integrated circuits (ASICs), application specific standards (ASSPs), system-on-a-chip (SOCs), complex programmable logic devices (CPLDs), and the like.

FIG. 8 illustrates a block diagram of an electronic device 800 in which one or more embodiments of the present disclosure may be implemented. It should be understood that the electronic device 800 illustrated in FIG. 8 is merely exemplary and should not constitute any limitation on the functionality and scope of the embodiments described herein. The electronic device 800 shown in FIG. 8 may be configured to implement the terminal device 110 in FIG. 1 or the apparatus 700 in FIG. 7.

As shown in FIG. 8, the electronic device 800 is in the form of a general-purpose computing device. Components of the electronic device 800 may include, but are not limited to, one or more processors or processing units 810, a memory 820, a storage device 830, one or more communication units 840, one or more input devices 850, and one or more output devices 860. The processing unit 810 may be an actual or virtual processor and capable of performing various processes according to programs stored in the memory 820. In multiprocessor system, multiple processing units execute computer-executable instructions in parallel to improve parallel processing capabilities of electronic device 800.

Electronic device 800 typically includes a plurality of computer storage media. Such media may be any available media accessible to the electronic device 800, including, but not limited to, volatile and non-volatile media, removable and non-removable media. The memory 820 may be volatile memory (e.g., registers, caches, random access memory (RAM)), non-volatile memory (e.g., read-only memory (ROM), electrically erasable programmable read-only memory (EEPROM), flash memory), or some combination thereof. Storage device 830 may be a removable or non-removable medium and may include a machine-readable medium, such as a flash drive, magnetic disk, or any other medium, which may be capable of storing information and/or data and may be accessed within electronic device 800.

The electronic device 800 may further include additional removable/non-removable, volatile/non-volatile storage media. Although not shown in FIG. 8, a disk drive for reading or writing from a removable, nonvolatile magnetic disk (e.g., a “floppy disk”) and an optical disk drive for reading or writing from a removable, nonvolatile optical disk may be provided. In these cases, each drive may be connected to a bus (not shown) by one or more data media interface. The memory 820 may include a computer program product 825 having one or more program modules configured to perform various methods or actions of various embodiments of the present disclosure.

The communication unit 840 is configured to communicate with another electronic device through a communication medium. Additionally, the functionality of components of the electronic device 800 may be implemented in a single computing cluster or multiple computing machines capable of communicating over a communication connection. Thus, the electronic device 800 may operate in a networked environment using logical connections with one or more other servers, network personal computers (PCs), or another network Node.

The input device 850 may be one or more input devices, such as a mouse, a keyboard, a trackball, or the like. The output device 860 may be one or more output devices, such as a display, a speaker, a printer, or the like. The electronic device 800 may also communicate with one or more external devices (not shown) through the communication unit 840 as needed, external devices such as storage devices, display devices, etc., communicate with one or more devices that enable a user to interact with the electronic device 800, or communicate with any device (e.g., a network card, a modem, etc.) that enables the electronic device 800 to communicate with one or more other electronic devices. Such communication may be performed via an input/output (I/O) interface (not shown).

According to example embodiments of the present disclosure, there is provided a computer-readable storage medium having computer-executable instructions stored thereon, wherein the computer-executable instructions are executed by a processor to implement the method described above. According to an exemplary embodiment of the present disclosure, a computer program product is further provided, the computer program product being tangibly stored on a non-transient computer readable medium and comprising computer executable instructions, the computer executable instructions being executed by a processor to implement the method described above.

Aspects of the present disclosure are described herein with reference to flowcharts and/or block diagrams of methods, apparatuses, devices, and computer program products implemented in accordance with the present disclosure. It should be understood that each block of the flowchart and/or block diagram, and combinations of blocks in the flowcharts and/or block diagrams, may be implemented by computer readable program instructions.

These computer-readable program instructions may be provided to a processing unit of a general purpose computer, special purpose computer, or other programmable data processing apparatus to produce a machine, such that the instructions, when executed by a processing unit of a computer or other programmable data processing apparatus, produce means to implement the functions/acts specified in the flowchart and/or block diagram. These computer-readable program instructions may also be stored in a computer-readable storage medium that cause the computer, programmable data processing apparatus, and/or other devices to function in a particular manner, such that the computer-readable medium storing instructions includes an article of manufacture including instructions to implement aspects of the functions/acts specified in the flowchart and/or block diagram (s).

The computer-readable program instructions may be loaded onto a computer, other programmable data processing apparatus, or other apparatus, such that a series of operational steps are performed on a computer, other programmable data processing apparatus, or other apparatus to produce a computer-implemented process such that the instructions executed on a computer, other programmable data processing apparatus, or other apparatus implement the functions/acts specified in the flowchart and/or block diagram block or blocks.

The flowchart and block diagrams in the figures show architecture, functionality, and operation of possible implementations of systems, methods, and computer program products according to various implementations of the present disclosure. In this regard, each block in the flowchart or block diagram may represent a module, program segment, or portion of an instruction that includes one or more executable instructions for implementing the specified logical function. In some alternative implementations, the functions noted in the blocks may also occur in a different order than noted in the figures. For example, two consecutive blocks may actually be performed substantially in parallel, which may sometimes be performed in the reverse order, depending on the functionality involved. It is also noted that each block in the block diagrams and/or flowchart, as well as combinations of blocks in the block diagrams and/or flowchart, may be implemented with a dedicated hardware-based system that performs the specified functions or actions, or may be implemented in a combination of dedicated hardware and computer instructions.

Various implementations of the present disclosure have been described above, which are exemplary, not exhaustive, and are not limited to the implementations disclosed. Many modifications and variations will be apparent to those of ordinary skill in the art without departing from the scope and spirit of the various implementations illustrated. The selection of the terms used herein is intended to best explain the principles of the implementations, the practical application, or improvements to the technology in the marketplace, or to enable others of ordinary skill in the art to understand the various embodiments disclosed herein.

I/We claim:

1. A method for information interaction, comprising:
in response to a consultation initiating operation, providing an interaction interface of a user and a digital assistant for a health consultation of the user;
determining, based on at least one round of conversation between the user and the digital assistant in the interaction interface, whether an information collection requirement for the health consultation is satisfied; and
in response to determining that the information collection requirement for the health consultation is satisfied, providing an anticipated health recommendation for the health consultation of the user at the interaction interface.

2. The method of claim 1, further comprising:
in response to determining that the information collection requirement for the health consultation is not satisfied, providing a guidance reply of the digital assistant to guide the user to input further information for the health consultation.

3. The method of claim 1, in the at least one round of conversation, further comprising:
in response to receiving a first query of the user, generating a first reply to the first query based at least on the first query; and
providing the first reply at the interaction interface.

4. The method of claim 3, wherein generating the first reply comprises:
generating a prompt input corresponding to the first query based at least on a prompt bank for the digital assistant;
providing the prompt input to a model to obtain an output of the model; and
determining the first reply based on the output of the model.

5. The method of claim 4, wherein determining the first reply based on the output of the model comprises:
determining whether the output of the model satisfies a predetermined medical verification rule; and
in response to the output of the model satisfying the medical verification rule, determining the first reply based on the output of the model.

6. The method of claim 5, further comprising:
in response to the output of the model failing to satisfy the medical verification rule, modifying the prompt input;
providing the modified prompt input to the model to obtain a further output of the model; and
determining the first reply based on the further output of the model.

7. The method of claim 1, further comprising:
providing at least one category label of a query example at the interaction interface; and
providing, based on a target category label in the at least one category label being selected, at least one query example under the target category label at the interaction interface.

8. The method of claim 1, wherein a reply of the digital assistant in the at least one round of conversation is used to guide the user to provide at least one class of health information.

9. The method of claim 1, wherein the model is trained based on a general corpus and a specialized corpus for medical field.

10. The method of claim 1, wherein providing the interaction interface comprises:
displaying identification information of a target consultation object in a predetermined area of the interaction interface, an object of the health consultation being the target consultation object.

11. The method of claim 10, further comprising:
in response to the consultation initiating operation, providing a confirmation request for the target consultation object; and
in response to receiving the confirmation for the target consultation object, determining that the object of the health consultation is the target consultation object.

12. An electronic device, comprising:
at least one processing unit; and
at least one memory coupled to the at least one processing unit and storing instructions for execution by the at least one processing unit, the instructions, when executed by the at least one processing unit, causing the device to perform acts comprising:
in response to a consultation initiating operation, providing an interaction interface of a user and a digital assistant for a health consultation of the user;
determining, based on at least one round of conversation between the user and the digital assistant in the interaction interface, whether an information collection requirement for the health consultation is satisfied; and
in response to determining that the information collection requirement for the health consultation is satisfied, providing an anticipated health recommendation for the health consultation of the user at the interaction interface.

13. The electronic device of claim 12, wherein the acts further comprises:
in response to determining that the information collection requirement for the health consultation is not satisfied, providing a guidance reply of the digital assistant to guide the user to input further information for the health consultation.

14. The electronic device of claim 12, in the at least one round of conversation, the acts further comprising:
in response to receiving a first query of the user, generating a first reply to the first query based at least on the first query; and
providing the first reply at the interaction interface.

15. The electronic device of claim 14, wherein generating the first reply comprises:
generating a prompt input corresponding to the first query based at least on a prompt bank for the digital assistant;
providing the prompt input to a model to obtain an output of the model; and
determining the first reply based on the output of the model.

16. The electronic device of claim 15, wherein determining the first reply based on the output of the model comprises:
determining whether the output of the model satisfies a predetermined medical verification rule; and
in response to the output of the model satisfying the medical verification rule, determining the first reply based on the output of the model.

17. The electronic device of claim 16, wherein the acts further comprises:
in response to the output of the model failing to satisfy the medical verification rule, modifying the prompt input;
providing the modified prompt input to the model to obtain a further output of the model; and
determining the first reply based on the further output of the model.

18. The electronic device of claim 12, wherein the acts further comprises:
providing at least one category label of a query example at the interaction interface; and
providing, based on a target category label in the at least one category label being selected, at least one query example under the target category label at the interaction interface.

19. The electronic device of claim 12, wherein a reply of the digital assistant in the at least one round of conversation is used to guide the user to provide at least one class of health information.

20. A non-transitory computer readable storage medium storing thereon a computer program which, when executed by a processor, implements a method for information interaction comprising:
in response to a consultation initiating operation, providing an interaction interface of a user and a digital assistant for a health consultation of the user;
determining, based on at least one round of conversation between the user and the digital assistant in the interaction interface, whether an information collection requirement for the health consultation is satisfied; and
in response to determining that the information collection requirement for the health consultation is satisfied, providing an anticipated health recommendation for the health consultation of the user at the interaction interface.
